(19) United States
(12) Patent Application Publication
(10) Pub. No.: US 2025/0367215 A1
(43) Pub. Date: Dec. 4, 2025

(54) PHARMACEUTICAL COMBINATION COMPRISING A CHOLANE DERIVATIVE AND A STATIN OR URSODESOXYCHOLIC ACID

(71) Applicant: BAR PHARMACEUTICALS SOCIETA' A RESPONSABILITA' LIMITATA, Reggio Nell'Emilia (IT)

(72) Inventors: Stefano Fiorucci, Reggio Nell'Emilia (IT); Angela Zampella, Reggio Nell'Emilia (IT)

(21) Appl. No.: 19/110,186

(22) PCT Filed: Sep. 12, 2023
(86) PCT No.: PCT/IB2023/059017

§ 371 Date: Mar. 10, 2025

(30) Foreign Application Priority Data
Sep. 13, 2022 (IT) 102022000018669

(51) Int. Cl. A61K 31/575; A61K 31/40; A61P 1/16

(52) U.S. Cl. CPC A61K 31/575; A61K 31/40; A61P 1/16

(57) ABSTRACT

The present invention relates to a pharmaceutical combination comprising 6α-ethyl-3α, 7 a-dihydroxy-24-nor-5 β-cholan-23-ol or a pharmaceutically acceptable salt thereof and a statin and to a combination comprising 6α-ethyl-3α, 7α-dihydroxy-24-nor-5β-cholan-23-ol and ursodesoxycholic acid and the use thereof for the treatment of non-alcoholic liver steatosis and non-alcoholic steatohepatitis.

### CROSS-REFERENCE TO RELATED APPLICATIONS

This patent application claims priority from Italian Patent Application No. 102022000018669 filed on Sep. 13, 2022, the entire disclosure of which is incorporated herein by reference.

### TECHNICAL FIELD

The present invention relates to pharmaceutical compositions comprising 6α-ethyl-3α,7α-dihydroxy-24-nor-5β-cholan-23-ol (BAR502).

### BACKGROUND OF THE INVENTION

Nonalcoholic fatty liver disease (NAFLD) represents the hepatic manifestation of the metabolic syndrome and is associated with metabolic abnormalities such as obesity, insulin resistance, fasting hyperglycaemia, dyslipidemia and altered adipokine profiles. Its worldwide prevalence continues to increase with the growing obesity epidemic becoming the most common cause of chronic liver disease in the last decade.

NAFLD is characterised by an excessive accumulation of lipids in the hepatocytes: in the early stages of the disease, simple hepatic steatosis is present, which may evolve into non-alcoholic steatohepatitis (NASH) and in more severe cases, liver fibrosis and cirrhosis may also occur with a consequent increased risk of developing hepatocellular carcinoma (HCC).

BAR502 has the following formula:

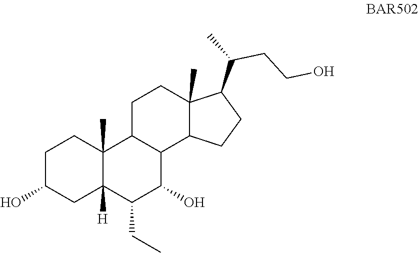 [SMILES: CC[C@H]1[C@@H](O)C2C3CC[C@H]([C@H](C)CCO)[C@@]3(C)CCC2[C@@]2(C)CC[C@@H](O)C[C@@H]12]

It is known from WO2015181275 to be a dual TGR5/GPBAR1 agonist that is used in the treatment of NAFLD.

Since NAFLD and NASH are highly widespread syndromes for which, however, no approved effective therapies are currently available, there is great interest in the scientific community towards the identification of new therapies.

### SUMMARY

Therefore, the aim of the present invention is to provide a new treatment for NAFLD and NASH.

This aim is achieved by a pharmaceutical combination according to claim 1, a use thereof according to claim 5, a combination according to claim 7 and a use thereof according to claim 8.

### BRIEF DESCRIPTION OF THE DRAWINGS

The present invention will now be described in detail with reference to the figures of the accompanying drawings, wherein:

FIG. 1 shows the change in body weight over time in C57BL6 mice following oral administration of only HFD-F, HFD-F+BAR502 30 mg/kg, HFD-F+atorvastatin 50 mg/kg or HFD-F and the combination BAR502 (30 mg/kg)+atorvastatin (50 mg/kg);

FIG. 2 shows the change in glucose over time in C57BL6 mice following oral administration of only HFD-F, HFD-F+BAR502 30 mg/kg, HFD-F+atorvastatin 50 mg/kg or HFD-F and the combination BAR502 (30 mg/kg)+atorvastatin (50 mg/kg);

FIG. 3 shows a) the levels of AST and b) the levels of ALT in the blood of C57BL6 mice measured following oral administration of only HFD-F, HFD-F+BAR502 30 mg/kg, HFD-F+atorvastatin 50 mg/kg or HFD-F and the combination BAR502 (30 mg/kg)+atorvastatin (50 mg/kg);

FIG. 4 shows the cholesterol levels in the blood of C57BL6 mice following oral administration of only HFD-F, HFD-F+BAR502 30 mg/kg, HFD-F+atorvastatin 50 mg/kg or HFD-F and the combination BAR502 (30 mg/kg)+atorvastatin (50 mg/kg);

FIG. 5 shows histological sections of liver following oral administration of only HFD-F, HFD-F+BAR502 30 mg/kg, HFD-F+atorvastatin 50 mg/kg or HFD-F and the combination BAR502 (30 mg/kg)+atorvastatin (50 mg/kg);

FIG. 6 shows the score of the hepatic steatosis on histological sections of liver referred to in FIG. 5;

FIG. 7 shows the score of the hepatic “ballooning” (ballooning degeneration) on histological sections of liver referred to in FIG. 5;

FIG. 8 shows the change in body weight over time in C57BL6 mice following oral administration of only HFD-F, HFD-F+BAR502 30 mg/kg, HFD-F+UDCA 30 mg/kg or HFD-F and the combination of BAR502 (30 mg/kg) and UDCA (30 mg/kg);

FIG. 9 shows the glucose change in the blood over time in C57BL6 mice following oral administration of only HFD-F, HFD-F+BAR502 30 mg/kg, HFD-F+UDCA 30 mg/kg or HFD-F and the combination of BAR502 (30 mg/kg) and UDCA (30 mg/kg);

FIG. 10 shows a) the levels of AST and b) the levels of ALT in the blood of C57BL6 mice measured following oral administration of only HFD-F, HFD-F+BAR502 30 mg/kg, HFD-F+UDCA 30 mg/kg or HFD-F and the combination of BAR502 (30 mg/kg) and UDCA (30 mg/kg);

FIG. 11 shows the cholesterol levels in the blood of C57BL6 mice following oral administration of only HFD-F, HFD-F+BAR502 30 mg/kg, HFD-F+UDCA 30 mg/kg or HFD-F and the combination of BAR502 (30 mg/kg) and UDCA (30 mg/kg);

FIG. 12 shows the histological analysis of liver sections following oral administration of only HFD-F, HFD-F+BAR502 30 mg/kg, HFD-F+UDCA 30 mg/kg or HFD-F and the combination of BAR502 (30 mg/kg) and UDCA (30 mg/kg);

FIG. 13 shows a) the score related to the ballooning degeneration (lipid deposition) in the various groups subject to treatment; b) the score related to the severity of steatosis in the various treatment groups.

### DESCRIPTION OF EMBODIMENTS

According to a first aspect of the invention there is provided a pharmaceutical combination comprising 6α-ethyl-3α,7α-dihydroxy-24-nor-5β-cholan-23-ol or a pharmaceutically acceptable salt thereof and a statin.

In one embodiment the statin is selected from the group consisting of atorvastatin, cerivastatin, fluvastatin, lovastatin, mevastatin, pitavastatin, pravastatin, rosuvastatin and simvastatin and preferably it is atorvastatin.

According to a further aspect of the present invention there is provided a pharmaceutical combination comprising 6α-ethyl-3α,7α-dihydroxy-24-nor-5β-cholan-23-ol or a pharmaceutically acceptable salt thereof and ursodesoxycholic acid or a pharmaceutically acceptable salt thereof.

The combinations according to the first and second aspect of the invention may further comprise at least one pharmacologically acceptable excipient.

The combinations of the invention may be included in pharmaceutical compositions and dosage units thereof and in such form may be used as solids, such as filled tablets or capsules, or liquids such as solutions, suspensions, emulsions, elixirs or capsules filled therewith, all for oral use or in the form of sterile injectable solutions for parenteral administration (including subcutaneous and intravenous use).

Such pharmaceutical compositions and the unit dosage forms thereof may comprise ingredients in conventional percentages, with or without additional compounds or active ingredients, and such unit dosage forms may comprise any suitable effective amount of each active ingredient commensurate with the intended daily dosage interval to be used.

The pharmaceutical compositions containing the combinations of the present invention can be prepared in a manner well known in the pharmaceutical technique. Generally, the combinations of the present invention are administered in a pharmaceutically effective amount. The amount of the combination actually administered will typically be determined by a physician, taking into account relevant circumstances, including the condition to be treated, the route of administration chosen, the actual combination administered, the age, the weight, and the response of the individual patient, the severity of the patient's symptoms, and the like.

The pharmaceutical compositions containing the combinations of the present invention can be administered by means of a number of routes including oral, rectal, subcutaneous, intravenous, intramuscular, intranasal, and pulmonary routes. The compositions for oral administration can take the form of liquid solutions or suspensions in bulk or in bulk powders. More commonly, however, the compositions are presented in unit dosage forms to facilitate the precise dosing. The expression “unit dosage forms” refers to physically distinct units suitable as unit dosages for human and other mammalian subjects, each unit containing a predetermined amount of active material calculated to produce the desired therapeutic effect, in association with an acceptable pharmaceutical excipient. The typical unit dosage forms include pre-filled, pre-dosed ampoules or syringes of the liquid compositions or pills, tablets, capsules or similar in the case of solid compositions.

The liquid forms suitable for oral administration may include a suitable aqueous or non-aqueous vehicle with buffering agents, suspending and dispersing agents, dyes, flavours and the like. The solid forms may include, for example, any of the following ingredients, or compounds of similar nature: a binder such as microcrystalline cellulose, tragacanth gum or gelatin; an excipient such as starch or lactose; a disintegrating agent such as alginic acid, Primogel or corn starch; a lubricant such as magnesium stearate; a flow agent such as colloidal silicon dioxide; a sweetening agent such as sucrose, lactose or saccharin; or a flavouring agent such as peppermint, methyl salicylate or orange flavouring.

The injectable compositions are typically based on sterile injectable solution or phosphate buffered solution or other injectable vehicles known in the art.

The pharmaceutical compositions may be in the form of tablets, pills, capsules, solutions, suspensions, emulsions, powders, suppositories and as sustained release formulations.

If desired, the tablets can be coated by standard aqueous or non-aqueous techniques. In some embodiments, such compositions and preparations may contain at least 0.1% of active compounds. The percentage of active compound in these compositions can be varied, of course, and can suitably be between about 1% and about 60% of the unit weight. The amount of active compound in such therapeutically useful compositions is such that the therapeutically active dosage will be obtained. The active compounds may also be administered intranasally like, for example, liquid drops or sprays.

The tablets, pills, capsules, and the like may also contain a binder such as tragacanth gum, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When a dosage unit form is a capsule, it may contain, in addition to the materials of the above type, a liquid carrier such as a fatty oil. Various other materials may be present as coatings or to modify the physical form of the dosage unit. For example, the tablets can be coated with shellac, sugar, or both. A syrup or elixir may contain, in addition to the active ingredient, sucrose as a sweetener, methyl and propyl parabens as preservatives, a dye and a flavouring agent such as cherry or orange flavour. To avoid breakage during the transit through the upper part of the gastrointestinal tract, the composition is an enteric-coated formulation.

The compositions for pulmonary administration include, but are not limited to, dry powder compositions consisting of the powdered active compounds and the powder of a suitable carrier and/or lubricant. The compositions for pulmonary administration may be inhaled from any suitable dry powder inhaler device known to the person skilled in the art.

The administration of the compositions is performed according to a protocol and at a dosage sufficient to reduce inflammation and pain in the subject. In some embodiments, the active ingredients in the pharmaceutical compositions are generally formulated in dosage units. The dosage unit may contain from 0.1 to 1000 mg of active compounds for each dosage unit per daily administration.

In some embodiments, the effective amounts for a specific formulation will depend on the severity of the disease, disorder or condition, the previous therapy, the health status of the individual and the response to the drug. In some embodiments the dose is in the range from 0.001% by weight to about 60% by weight of the formulation.

With regard to the formulations relative to any type of route of administration, methods and formulations for the administration of drugs are described in Remington's Pharmaceutical Sciences, 17th Edition, Gennaro et al. Ed., Mack Publishing Co., 1985 and Remington's Pharmaceutical Sciences, Gennaro AR ed. 20th Edition, 2000, Williams & Wilkins PA, USA and Remington: The Science and Practice of Pharmacy, 21th Edition, Lippincott Williams & Wilkins Ed., 2005; and in Loyd V. Allen and Howard C. Ansel, Ansel's Pharmaceutical Dosage Forms and Drug Delivery Systems, 10th Edition, Lippincott Williams & Wilkins Ed., 2014.

The components described above for orally administered or injectable compositions are representative only.

The combination of the present invention may also be administered in sustained release forms or by sustained release drug delivery systems.

Alternatively, the active ingredients of the combinations of the present invention, although not formulated in a single pharmaceutical formulation, may be administered jointly or independently at the same time or separately at intervals.

According to a further aspect of the present invention, the above-described pharmaceutical combinations may be used for the treatment of a disorder selected from the group consisting of non-alcoholic hepatic steatosis and non-alcoholic steatohepatitis.

### DESCRIPTION OF EMBODIMENTS

In the following, the present invention will be shown by means of some examples, which are not intended to be considered limiting of the scope of the invention.

### Example 1. Efficacy of the Pharmaceutical Combination Comprising BAR502 and Atorvastatin

Several pre-clinical NASH models are available. Among the mouse models, steatohepatitis induced by long-term administration of a high-fat (HFD) and fructose (F) diet leading to the development of steatosis, inflammation and fibrosis, shows the best correlation with what can be observed in the human disease. Using this mouse model, the inventors investigated the efficacy of the association between BAR502 and atorvastatin in preventing the development of NASH.

### Method

12-week-old C57BL6 mice were fed a diet containing 60% of calories from fat, and fructose added in drinking water (42 g/l) (HFD-F), or control diet, for 61 days. The mice were randomized to receive, starting on day 6, only HFD-F, HFD-F+BAR502 30 mg/kg, HFD-F+atorvastatin 50 mg/kg or HFD-F and the combination BAR502 (30 mg/kg)+atorvastatin (50 mg/kg) by oral administration.

### Results

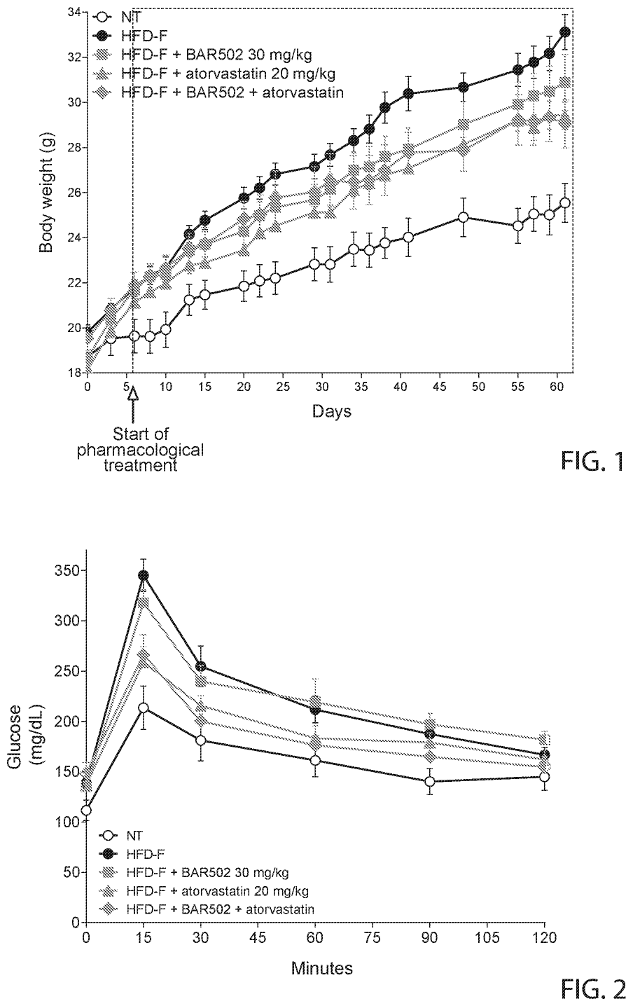

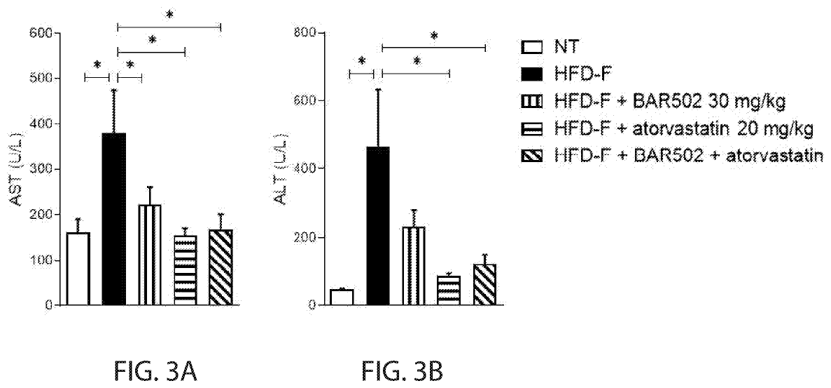

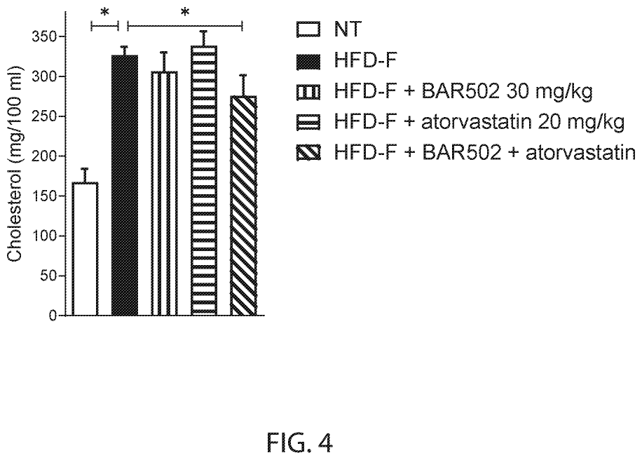

The results obtained in this mouse model show, surprisingly, that the combination of BAR502 and atorvastatin exerts a much greater beneficial effect than the single treatments. All three treatments show a beneficial effect on body weight gain (FIG. 1) but the association between BAR502 and atorvastatin shows strongly higher beneficial effects than the individual treatments in reducing insulin resistance (FIG. 2) as shown by the glucose load curve (OGTT) results. This data item is very interesting because insulin resistance represents one of the main characteristics of the disease in humans. Transaminase analysis (FIGS. 3a and 3b) showed that all three drug treatments effectively reduce the hepatic damage while hypercholesterolemia is significantly reduced only by the association between BAR502 and atorvastatin (FIG. 4). This effect exerted by the combination of the treatments alone is of high interest because the patients with NASH show an increase in the blood concentration of cholesterol which represents one of the main risk factors for the development of vascular diseases.

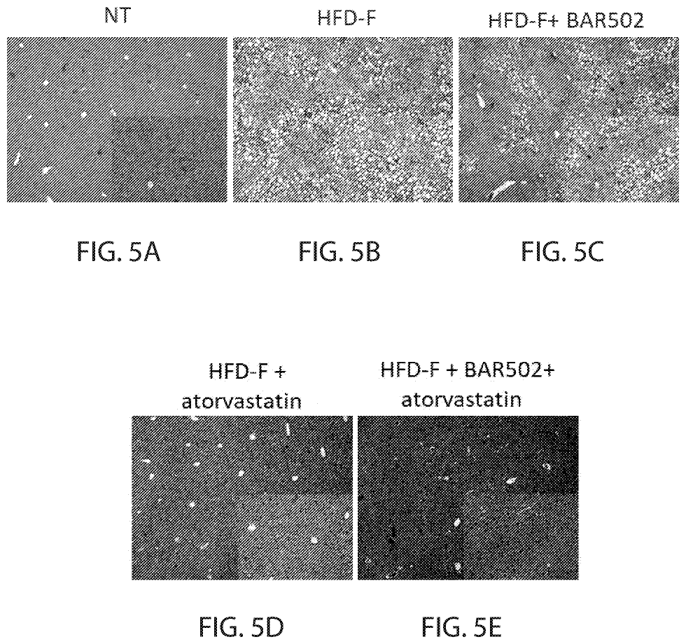

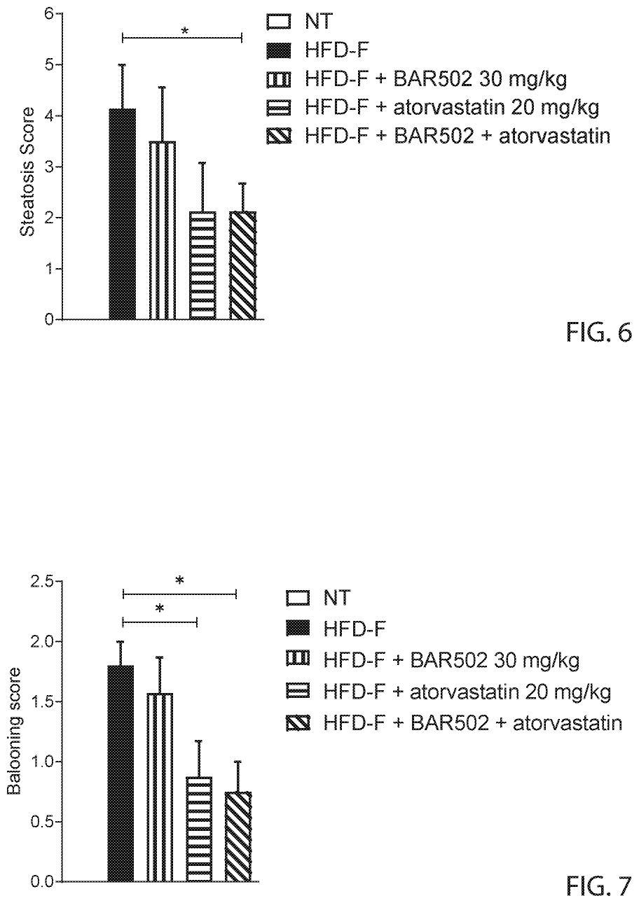

The main feature of NAFLD is lipid deposition at the hepatic level leading first to steatosis and then to steatohepatitis. The histological analysis (FIGS. 5A-5E) of this aspect of the disease in the mouse model used showed that the HFD-F diet induces high lipid deposition at the hepatic level with consequent hepatocyte ballooning and cell death (as attested by the increase in AST and ALT values in this experimental group). Surprisingly, the association between BAR502 and atorvastatin completely prevents lipid deposition in the hepatocytes, as demonstrated by the histologies and steatosis score (FIG. 6), thus protecting the hepatocytes from ballooning (FIG. 7) and from the consequent liver damage.

### Example 2. Efficacy of the Pharmaceutical Combination Comprising BAR502 and Ursodesoxycholic Acid (UDCA)

The mouse model of Example 1 was used once again. Using this mouse model, the inventors therefore investigated the effectiveness of the association between BAR502 and UDCA in preventing the development of NASH.

### Method

12-week-old C57BL6 mice were fed a diet containing 60% of calories from fat, and fructose added in drinking water (42 g/l) (HFD-F), or control diet, for 61 days. The mice were randomized to receive, starting on day 6, only HFD-F, HFD-F+BAR502 30 mg/kg, HFD-F+UDCA 30 mg/kg, or HFD-F and the combination of BAR502 (30 mg/kg) and UDCA (30 mg/kg) by oral administration.

### Results

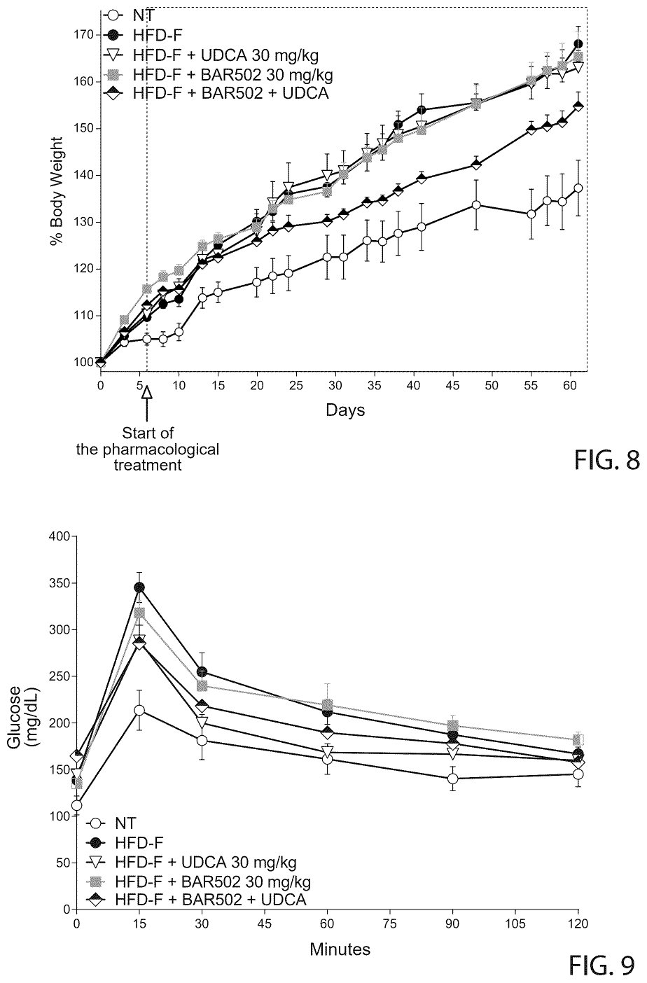

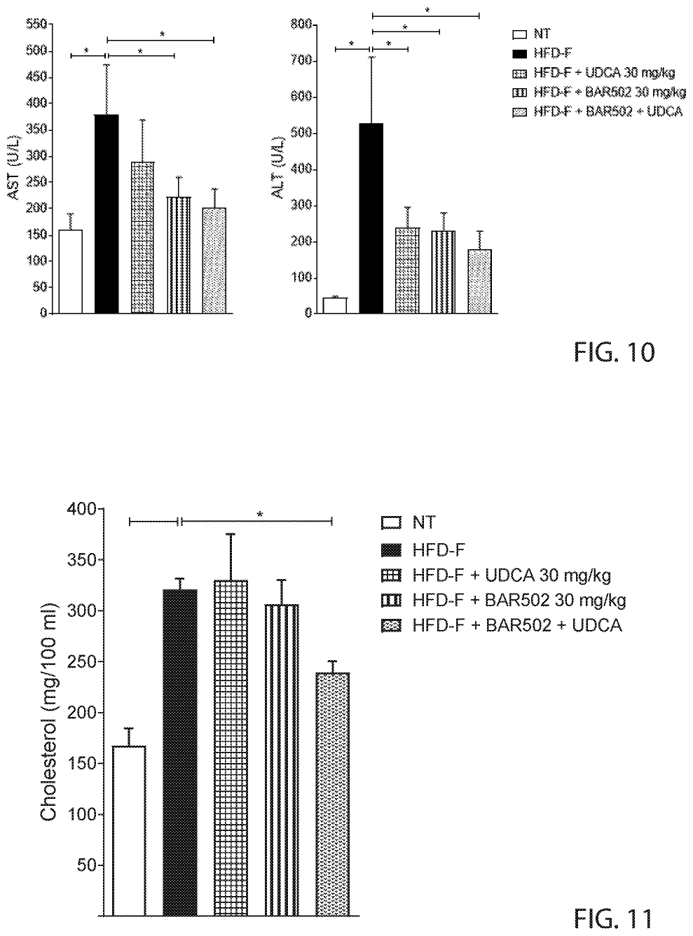

The results obtained in this mouse model show, surprisingly, that the combination of BAR502 and UDCA exerts a much greater beneficial effect than the single treatments. For example, the combination of BAR502 and UDCA alone shows a significant beneficial effect on body weight gain (FIG. 8) while beneficial effects on insulin resistance, as shown by the results of the glucose load curve (OGTT), are exerted, in addition to the combination, also by the single treatment with UDCA (FIG. 9). This data item is very interesting because insulin resistance represents one of the main characteristics of human disease. The transaminase analysis (FIG. 10) showed that all three drug treatments effectively reduce hepatic damage while hypercholesterolemia (FIG. 11) is significantly reduced only by the combination of BAR502 and UDCA. This effect exerted by the combination alone of the treatments is of high translational/clinical interest because the patients with NASH show an increase in the blood concentration of cholesterol which represents one of the main risk factors for the development of vascular diseases.

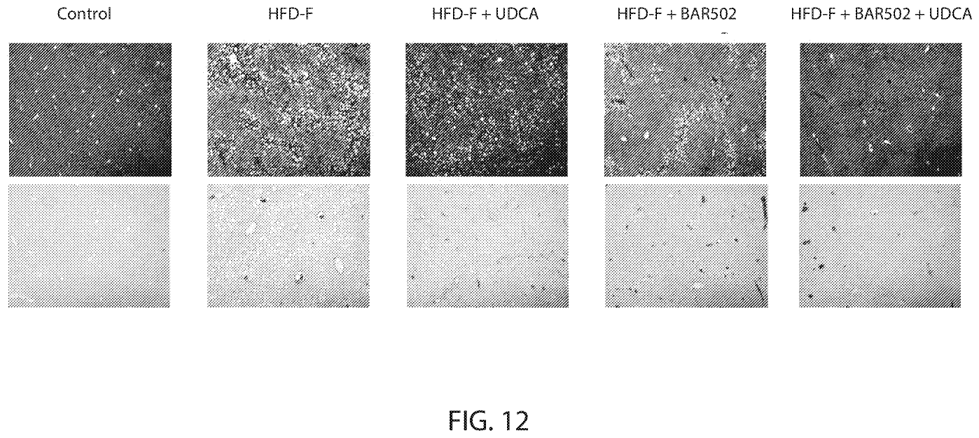

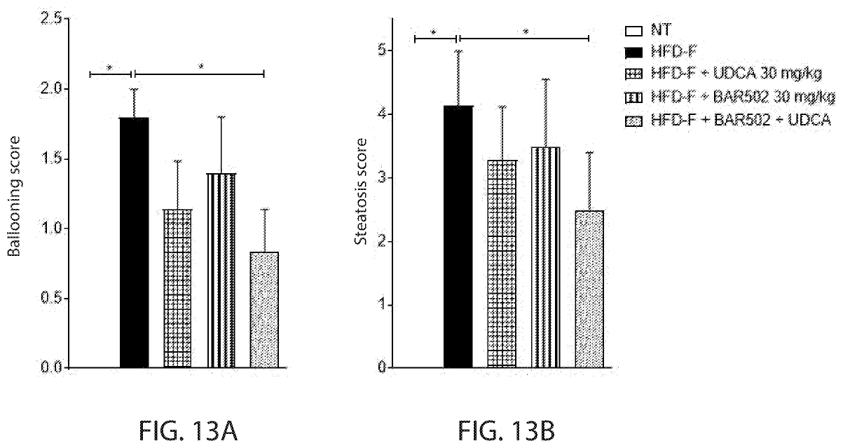

The main feature of NAFLD is lipid deposition at the hepatic level leading first to steatosis and then to steatohepatitis. The histological analysis of this aspect of the disease in the mouse model used showed that the HFD-F diet induces high lipid deposition at the hepatic level with consequent hepatocyte ballooning and cell death (as attested by the increase in AST and ALT values in this experimental group) and liver fibrosis. Surprisingly, the association between BAR502 and UDCA, with a much higher effect than that exerted by the individual treatments, prevents lipid deposition in the hepatocytes, as demonstrated by the histologies (FIG. 12) and by the steatosis score (FIG. 13), thus protecting the hepatocytes from ballooning and consequent liver damage and fibrosis, which represents one of the most fearsome complications of the human disease.

1. A pharmaceutical combination comprising 6α-ethyl-3α,7α-dihydroxy-24-nor-5β-cholan-23-ol or a pharmaceutically acceptable salt thereof and a statin.

2. The pharmaceutical combination according to claim 1, characterized in that said statin is selected from the group consisting of atorvastatin, cerivastatin, fluvastatin, lovastatin, mevastatin, pitavastatin, pravastatin, rosuvastatin and simvastatin.

3. The pharmaceutical combination according to claim 2, characterized in that said statin is atorvastatin.

4. The pharmaceutical combination according to claim 1, further comprising at least one pharmacologically acceptable excipient.

5. A method for the treatment of a disorder selected from the group consisting of non-alcoholic hepatic steatosis and non-alcoholic steatohepatitis in a patient in need thereof by administering a pharmaceutical combination according to claim 1.

6. The method according to claim 5 wherein the pharmaceutical combination is in the form of a kit of parts for combined administration, in which said 6α-ethyl-3α,7α-dihydroxy-24-nor-5β-cholan-23-ol, or a pharmaceutically acceptable salt thereof, and said statin, are administered jointly or independently at the same time or separately at intervals.

7. A pharmaceutical combination comprising 6α-ethyl-3α,7α-dihydroxy-24-nor-5β-cholan-23-ol or a pharmaceutically acceptable salt thereof and ursodesoxycholic acid or a pharmaceutically acceptable salt thereof.

8. The pharmaceutical combination according to claim 7, further comprising at least one pharmacologically acceptable excipient.

9. A method for the treatment of a disorder selected from the group consisting of non-alcoholic hepatic steatosis and non-alcoholic steatohepatitis in a patient in need thereof by administering a pharmaceutical combination according to claim 7.

10. The method according to claim 9 wherein the pharmaceutical combination is in the form of a kit of parts for combined administration, in which said 6α-ethyl-3α,7α-dihydroxy-24-nor-5β-cholan-23-ol, or a pharmaceutically acceptable salt thereof, and said ursodeoxycholic acid or a pharmaceutically acceptable salt thereof, are administered jointly or independently at the same time or separately at intervals.
